(19) United States
(12) Patent Application Publication
(10) Pub. No.: US 2022/0387554 A1
(43) Pub. Date: Dec. 8, 2022

(54) TREATMENTS FOR EXUDATIVE MACULOPATHIES

(71) Applicant: Mark H. Nelson, Winston-Salem, NC (US)

(72) Inventor: Mark H. Nelson, Winston-Salem, NC (US)

(21) Appl. No.: 17/828,784

(22) Filed: May 31, 2022

Related U.S. Application Data
(60) Provisional application No. 63/196,877, filed on Jun. 4, 2021.

(51) Int. Cl. A61K 38/17; A61P 27/02; A61K 9/00; A61K 39/395

(52) U.S. Cl. CPC A61K 38/179; A61P 27/02; A61K 9/0019; A61K 9/0048; A61K 39/3955

(57) ABSTRACT

The present application provides methods for treating exudative maculopathies including methods based on rapid centrifugal fluidic immunoassays.

### I. CROSS-REFERENCE TO RELATED APPLICATIONS

This application claims benefit of U.S. Provisional Application No. 63/196,877, filed Jun. 4, 2021, the contents of which are hereby incorporated by reference.

### II. BACKGROUND OF THE INVENTION

Retinal vein occlusion (RVO) is the second most common retinal vascular disease after diabetic retinopathy, and presents in two ways—branch retinal vein occlusion (BRVO) and central retinal vein occlusion (CRVO) with BRVO being four times more common as CRVO. The pathophysiology of RVO involves the intraluminal thrombus formation, which may be associated with systemic conditions such as hypertension, hyperlipidemia, diabetes and abnormal thrombophilia. The blockage of venous circulation causes an elevation of intraluminal pressure in the capillaries, leading to hemorrhages and leakage of fluid within the retina. There is associated reduction of retinal perfusion as well, which leads to the secretion of vascular endothelial growth factor (VEGF). VEGF therefore has a leading role in RVO and leads to the clinical finding of macular edema which limits vision.

The current treatment for RVO is anti-VEGF therapy, injected into the vitreous, which has largely replaced macular photocoagulation. Randomized controlled trials (VIBRANT for BRVO and GALILEO/COPERNICUS for CRVO) have shown efficacy and safety with aflibercept (Eylea) injections.

There is a need to further refine RVO treatments by reducing the amount of administered inappropriate drug levels not correlated to the current disease state in an individual.

### III. SUMMARY OF THE INVENTION

The inventor has determined that measuring VEGF, or other marker levels, in the clinic at the point of treatment and administering therapy based on the level of VEGF within a short time frame (i.e., during the same patient visit to the clinic) reduces inappropriate treatment and improves differentiated treatments, whether the paradigm is, e.g., initiating therapy with anti-VEGF or another inhibitor, and/or maintenance therapy which continues to suppress VEGF production.

A method of treating an exudative maculopathy in subject is provided comprising:

a) obtaining a sample of anterior chamber fluid from an anterior chamber of the subject's eye;
b) determining the level of vascular endothelial growth factor (VEGF) in the sample;
c) comparing the level of VEGF in the sample with a predetermined reference level of VEGF; and
d) administering to the eye of the subject intraocularly either an exudative maculopathy therapy A or an exudative maculopathy therapy B based on whether the level of VEGF in the sample is higher or not than the predetermined reference level therefor,
wherein if the level of the VEGF is higher than the predetermined reference level therefor then exudative maculopathy therapy A is administered and wherein if the level of the VEGF is not higher than the predetermined reference level therefor then exudative maculopathy therapy B is administered,
wherein exudative maculopathy therapy A is an anti-VEGF biologic or anti-VEGF small molecule, and
wherein exudative maculopathy therapy B is a either an anti-intercellular adhesion molecule 1 (ICAM1), anti-interleukin 8 (IL8), anti-interleukin 6 (IL6), anti-monocyte chemoattractant protein 1 (MCP1), anti-interleukin 1 alpha or beta (IL1 alpha/beta), anti-platelet-derived growth factor (PDGF), or anti-tumor necrosis factor alpha (TNF-alpha), or anti-interleukin-12 subunit p40 (IL12P40), and/or anti-angiopoietin (ANG2) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule, and
   wherein steps b) through d) are performed within 2 hours of step a).

A method of treating an exudative maculopathy in subject comprising:
i) obtaining a sample of anterior chamber fluid from an anterior chamber of the subject's eye;
ii) determining the level of one or more of the following molecules:
   vascular endothelial growth factor (VEGF),
   intercellular adhesion molecule 1 (ICAM1),
   interleukin 6/8 (IL6/8),
   monocyte chemoattractant protein (MCP1),
   interleukin 1 alpha or beta (IL1 alpha/beta),
   platelet-derived growth factor (PDGF),
   tumor necrosis factor alpha (TNF-alpha),
   interleukin-12 subunit p40 (IL12P40),
   angiopoietin (ANG2),
iii) comparing the level of the one or more molecules with a predetermined reference level for each, respectively,
iv) administering to the eye of the subject intraocularly either an exudative maculopathy therapy A or an exudative maculopathy B based on whether the level of the one or more molecules is higher or not than the predetermined reference level for each, respectively, wherein if the level of the one or more molecules is higher than the predetermined reference level therefor then exudative maculopathy therapy A is administered and wherein if the level of the one or more molecules is not higher than the predetermined reference level therefor then exudative maculopathy therapy B is administered,
   wherein when the molecule is VEGF then therapy A is an anti-VEGF biologic or anti-VEGF small molecule,
   and wherein therapy B is a either an anti-intercellular adhesion molecule 1 (ICAM1), anti-interleukin 6/8 (IL6/8), anti-monocyte chemoattractant protein (MCP1), anti-interleukin 1 alpha or beta (IL1 alpha/beta), anti-platelet-derived growth factor (PDGF), anti-tumor necrosis factor alpha (TNF-alpha), anti-interleukin-12 subunit p40 (IL12P40), and/or anti-angiopoietin (ANG2) biologic or small molecule, or, wherein the subject has previously been administered a dose of an anti-VEGF biologic or anti-VEGF small molecule, is a lower dose of the anti-VEGF biologic or anti-VEGF small molecule,
      wherein steps ii) through iv) are performed within 2 hours of step i).

### IV. DETAILED DESCRIPTION

The inventor has determined that measuring VEGF, or other marker levels, in the clinic at the point of treatment and administering therapy based on the level of VEGF within a short time frame (i.e., during the same patient visit to the clinic) reduces inappropriate treatment and improves differentiated treatments, whether the paradigm is, e.g., initiating therapy with anti-VEGF or another inhibitor, and/or maintenance therapy which continues to suppress VEGF production.

A method of treating an exudative maculopathy in subject is provided comprising:

a) obtaining a sample of anterior chamber fluid from an anterior chamber of the subject's eye;
b) determining the level of vascular endothelial growth factor (VEGF) in the sample;
c) comparing the level of VEGF in the sample with a predetermined reference level of VEGF; and
d) administering to the eye of the subject intraocularly either an exudative maculopathy therapy A or an exudative maculopathy therapy B based on whether the level of VEGF in the sample is higher or not than the predetermined reference level therefor,
wherein if the level of the VEGF is higher than the predetermined reference level therefor then exudative maculopathy therapy A is administered and wherein if the level of the VEGF is not higher than the predetermined reference level therefor then exudative maculopathy therapy B is administered,
wherein exudative maculopathy therapy A is an anti-VEGF biologic or anti-VEGF small molecule, and
wherein exudative maculopathy therapy B is a either an anti-intercellular adhesion molecule 1 (ICAM1), anti-interleukin 8 (IL8), anti-interleukin 6 (IL6), anti-monocyte chemoattractant protein 1 (MCP1), anti-interleukin 1 alpha or beta (IL1 alpha/beta), anti-platelet-derived growth factor (PDGF), or anti-tumor necrosis factor alpha (TNF-alpha), or anti-interleukin-12 subunit p40 (IL12P40), and/or anti-angiopoietin (ANG2) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule, and
wherein steps b) through d) are performed within 2 hours of step a).

In embodiments, the exudative maculopathy is a retinal vein occlusion and exudative maculopathy therapy B is a either an anti-intercellular adhesion molecule 1 (ICAM1), anti-interleukin 8 (IL8), anti-interleukin 6 (IL6), anti-monocyte chemoattractant protein 1 (MCP1) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

In embodiments, the exudative maculopathy is a diabetic retinopathy and exudative maculopathy therapy B is a either an anti-intercellular adhesion molecule 1 (ICAM1), anti-interleukin 6 (IL6), anti-monocyte chemoattractant protein 1 (MCP1), anti-interleukin 1 alpha or beta (IL1 alpha/beta), anti-tumor necrosis factor alpha (TNF-alpha), and/or anti-angiopoietin (ANG2) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

In embodiments, the subject is experiencing macular bleeding and the treatment reduces the bleeding.

In embodiments, the exudative maculopathy is an exudative age-related macular degeneration and exudative maculopathy therapy B is a either an anti-platelet-derived growth factor (PDGF), anti-interleukin-12 subunit p40 (IL12P40), anti-intercellular adhesion molecule 1 (ICAM1), anti-monocyte chemoattractant protein 1 (MCP1) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

In embodiments, the subject is experiencing macular leakage and the treatment reduces the leakage.

A method is provided of treating an exudative maculopathy in subject comprising:

i) obtaining a sample of anterior chamber fluid from an anterior chamber of the subject's eye;
ii) determining the level of one or more of the following molecules:
vascular endothelial growth factor (VEGF),
intercellular adhesion molecule 1 (ICAM1),
interleukin 6/8 (IL6/8),
monocyte chemoattractant protein (MCP1),
interleukin 1 alpha or beta (IL1 alpha/beta),
platelet-derived growth factor (PDGF),
tumor necrosis factor alpha (TNF-alpha),
interleukin-12 subunit p40 (IL12P40),
angiopoietin (ANG2),
iii) comparing the level of the one or more molecules with a predetermined reference level for each, respectively,
iv) administering to the eye of the subject intraocularly either an exudative maculopathy therapy A or an exudative maculopathy B based on whether the level of the one or more molecules is higher or not than the predetermined reference level for each, respectively,
wherein if the level of the one or more molecules is higher than the predetermined reference level therefor then exudative maculopathy therapy A is administered and wherein if the level of the one or more molecules is not higher than the predetermined reference level therefor then exudative maculopathy therapy B is administered,
wherein when the molecule is VEGF then therapy A is an anti-VEGF biologic or anti-VEGF small molecule,
and wherein therapy B is a either an anti-intercellular adhesion molecule 1 (ICAM1), anti-interleukin 6/8 (IL6/8), anti-monocyte chemoattractant protein (MCP1), anti-interleukin 1 alpha or beta (IL1 alpha/beta), anti-platelet-derived growth factor (PDGF), anti-tumor necrosis factor alpha (TNF-alpha), anti-interleukin-12 subunit p40 (IL12P40), and/or anti-angiopoietin (ANG2) biologic or small molecule, or, wherein the subject has previously been administered a dose of an anti-VEGF biologic or anti-VEGF small molecule, is a lower dose of the anti-VEGF biologic or anti-VEGF small molecule,
wherein steps ii) through iv) are performed within 2 hours of step i).

In embodiments, the level of the VEGF or of the molecule in the sample is determined using a rapid ELISA.

In embodiments, the VEGF or of the molecule in the sample is determined using a rapid centrifugal fluidic immunoassay.

In embodiments, steps b) through d) are performed within 1 hour of step a).

In embodiments, steps b) through d) are performed within 0.5 hours of step a).

In embodiments, steps ii) through iv) are performed within 1 hour of step i).

In embodiments, steps ii) through iv) are performed within 0.5 hours of step i).

In embodiments, steps b) through d) are performed within 0.25 hours of step a).

In embodiments, steps ii) through iv) are performed within 0.25 hours of step i).

In embodiments, the retinal vein occlusion is branch retinal vein occlusion (BRVO).

In embodiments, the retinal vein occlusion is central retinal vein occlusion (CRVO).

In embodiments, the intraocular administration is by intravitreal injection.

Treating patients with RVO with standard of care anti-VEGF drugs (or inhibitors of other cytokines) and then correlating levels of VEGF in the ocular fluids to disease state manifested by retinal, specifically macular, thickening, as well as other biomarkers, allows a refined, improved treatment for patients.

Anterior chamber fluid VEGF levels will be measured by ELISA as well as other very rapid procedures, e.g., a rapid centrifugal fluidic immunoassay. VEGF (or cytokine) levels are determined within minutes, i.e., at the point-of-contact, and subsequently influence the treatment administered within the same visit to the physician, e.g., frequency, dose, and/or pharmaceutical category of drug therapy applied.

Retinal vein occlusion (RVO) is the second most common retinal vascular disease after diabetic retinopathy, and presents in two ways—branch retinal vein occlusion (BRVO) and central retinal vein occlusion (CRVO) with BRVO being four times more common as CRVO. The pathophysiology of RVO involves the intraluminal thrombus formation, which may be associated with systemic conditions such as hypertension, hyperlipidemia, diabetes and abnormal thrombophilia. The blockage of venous circulation causes an elevation of intraluminal pressure in the capillaries, leading to hemorrhages and leakage of fluid within the retina. There is associated reduction of retinal perfusion as well, which leads to the secretion of vascular endothelial growth factor (VEGF). VEGF therefore has a leading role in RVO and leads to the clinical finding of macular edema which limits vision.

The current treatment for RVO is anti-VEGF therapy, injected into the vitreous, which has largely replaced macular photocoagulation. Randomized controlled trials (VIBRANT for BRVO and GALILEO/COPERNICUS for CRVO) have shown efficacy and safety with aflibercept (Eylea) injections.

There is a need to further refine RVO treatments by reducing the amount of administered inappropriate drug levels not correlated to the current disease state in an individual.

The inventor has determined that measuring VEGF, or other marker levels, in the clinic at the point of treatment and administering therapy based on the level of VEGF within a short time frame (i.e., during the same patient visit to the clinic) reduces inappropriate treatment and improves differentiated treatments, whether the paradigm is, e.g., initiating therapy with anti-VEGF or another inhibitor, and/or maintenance therapy which continues to suppress VEGF production.

Treating patients with RVO with standard of care anti-VEGF drugs (or inhibitors of other cytokines) and then correlating levels of VEGF in the ocular fluids to disease state manifested by retinal, specifically macular thickening, as well as other biomarkers, allows a refined, improved treatment for patients.

In embodiments, the condition is treated with intravitreal injections of anti-VEGF drugs. In embodiments, the drug is brolucizumab, aflibercept, ranibizumab, bevacizumab, or pegaptanib. In embodiments, the condition is treated with an intravitreal steroid (e.g., triamcinolone acetonide or dexamethasone implant (Ozurdex)). In embodiments, the condition is diabetic macular edema and is treated with a long-lasting three year steroid (Iluvien). In embodiments, the condition is treated with Photodynamic Therapy Laser with Visudyne. In embodiments, the condition is treated with thermal laser (argon laser) which cauterized the leakage sites.

### Results

### EXAMPLE

Anterior chamber fluid VEGF levels will be measured by ELISA as well as other very rapid procedures, e.g., a rapid centrifugal fluidic immunoassay. VEGF (or cytokine) levels are determined within minutes, i.e., at the point-of-contact, and subsequently influence the treatment administered within the same visit to the physician, e.g., frequency, dose, and/or pharmaceutical category of drug therapy applied.

The following biomarkers will be factored into a pharmacodynamic model to determine the proper amount and frequency of anti-VEGF therapy.

Foveal thickness, in mm, known as Central Subfield Thickness, as objectively measured by Optical Coherent Tomography (OCT)

Macular volume, in cubic mm, as objectively measured by OCT.

Degree of retinal ischemia, as noted on intravenous fluorescein angiography (IVFA)

VEGF levels in anterior chamber fluid

Visual acuity, in ETDRS letters, best corrected

Rate of change of foveal thickness, in mm.

Rate of change macular edema, in cubic mm.

One way to measure VEGF levels in a sample obtained from the subject within a sufficiently rapid time is by a centrifugal fluidic immunoassay. For example, the Spin Dx® prototype centrifugal fluidic immunoassay (Sandia National Laboratories) involves specific technology that combines sedimentation principles applied to microspheres under centrifugal force with signal amplification using an enzyme and a fluorogenic substrate for readout. The simple single channel per assay platform separates, washes and concentrates antibody-coated microspheres from excess label to produce a sensitive fluorogenic response proportional to the amount of VEGF in the sample. It takes 15 minutes to obtain the results, therefore, the results are at the point-of-contact in the clinical setting.

Rapid measurement of VEGF level results can subsequently influence the treatment schedule by creating a dose-response curve that can predict the degree and timing of VEGF suppression via anti-VEGF medications.

Subjects will receive standard of care medications over the course of 52 weeks while undergoing a paracentesis prior to treatment at each study visit. Anterior chamber fluid removed from the eye during the paracentesis will be used to measure VEGF levels. VEGF levels will be used along with OCT imaging, VA, and IVFA to determine the frequency of future injections. High levels of VEGF and persistence of measured macular thickness on OCT imaging will result in the continuation of every 4 week injections until the macular edema resolves and/or the VEGF levels are reduced. Low levels of VEGF and resolution of measured macular thickness on OCT imaging will result in an extension of treatment frequency to every 8 weeks. These subjects will have their treatment window extended to 12 weeks if retinal leakage does not recur and if the VEGF levels measured remain low. Dosing windows will not be extended past 12 weeks.

The anterior chamber fluid taken at each visit will be divided for both the ELISA and Spin Dx analysis, after which any leftover fluid will be destroyed.

Recruitment will occur in an outpatient Ophthalmology clinic as patients are receiving eye exams for ocular related issues. Patients that are diagnosed with BRVO or CRVO will be asked by the Principle Investigator if they are interested in learning more about a research study related to their diagnosis. Patients that show interest will be presented with the study consent and the study will be reviewed with the patients. During this time patients will be provided time to review the consent and discuss the study with their families, friends or health care providers. Questions will be answered and any concerns will be addressed prior to obtaining consent. Patients will be informed that if they decide not to participate they will still receive treatment for any eye related issues. Other treatment options will be discussed with the patient by the Principal Investigator.

Patient data will not be collected for study related purposed until informed consent is obtained. Any data used to contact potential subjects prior to obtaining consent will be maintained in the electronic medical record. Informed consent will be administered prior to any study related activities being performed. During the screening phase medical history will be reviewed to determine eligibility. All eligibility criteria must be met before a patient can move forward with study treatment and testing of VEGF levels. Patients that do not meet enrollment criteria (screen-failed patients) will be offered standard of care treatment.

Study assessments, procedures and standard of care treatment will be started on Day 1 for patients successfully enrolled into the clinical trial and continue through week 52. Study visits will continue every 4, 8 or 12 weeks as determined by VEGF levels and macular thickness on OCT imaging. Treatment windows will not extend past 12 weeks. If a patient is experiencing an ocular issue they will be instructed to contact the study doctor to determine if an additional follow up visit is needed.

Subject Selection Criteria: This is a non-randomized study that will enroll approximately 10 subjects; 5 subjects with BRVO and 5 subjects with CRVO. Inclusion Criteria are:

1. Willingness and ability to provide written informed consent
2. Age >18 years
3. Diagnosis of Retinal Vein Occlusion with macular edema and central foveal thickness of >300 microns confirmed by intravenous fluorescein angiography and Optical Coherent Tomography
4. Visual Acuity between 20/25 and Hand Motion.

Exclusion Criteria are:

### 1. Bilateral Retinal Vein Occlusion

2. History of myocardial infraction, ischemia, or cerebrovascular accident within 6 weeks of screening.
3. Concurrent Proliferative Diabetic Retinopathy and/or Maculopathy

### 4. Concurrent Exudative Age-Related Macular Degeneration

5. Concurrent optic neuropathy with the presence of an afferent pupillary defect.
6. Previous vitrectomy in the study eye.
7. Currently pregnant or planning to become pregnant during the duration of the study. Women currently breastfeeding are also excluded.
8. Previous treatment for retinal vein occlusion in the study eye.
9. Any current medical condition which, in the opinion of the investigator, is considered to be uncontrolled.
10. History of allergy or hypersensitivity to study treatment, fluorescein, or any study procedure and treatment related ingredients (e.g. topical anesthetics, betadine, etc.)

Study Treatment: The recommended dose of aflibercept for the treatment of RVO is 2 mg (0.05 mL). All subjects will be administered an intravitreal injection of 2 mg (0.05 mL) aflibercept in a single-dose pre-filled syringe. All intravitreal injections will be performed by the Principle Investigator.

Treatment will be administered every 4 weeks until VEGF levels are brought into a range between 50 and 5000 picograms/ml, and macular thickness on OCT imaging is <300 microns at which time treatment will be administered every 8 weeks. If VEGF levels remain with study outlined range and macular thickness measurements remain below 300 microns, treatment will be extended to every 12 weeks.

Study drug will be stored between 2° C. to 8° C. until time of administration. Study treatment will be discontinued for the following reasons:

Pregnancy

The subject experiences a clinically significant or serious adverse event that would not be in align with the continuation in the study as determined by the Investigator

Study Assessments and Procedures: During the screening phase, after consent has been obtained, a complete medical history, including clinically significant diseases, chronic and ongoing conditions, and all current medications will be collected.

Blood pressure will be collected prior to the paracentesis and intravitreal injection.

Ocular assessments will be performed on both eyes at each study visit and will include:

1. Best Corrected Visual Acuity (BCVA) will be assessed using Snellen charts. If a subject's visual acuity is so poor that the subject can't see any letters on the charts, the number of fingers or hand motions will be checked.
2. Intraocular Pression (IOP) in mm HG will be measured prior to the paracentesis.
3. Slit lamp biomicroscopy will be performed to examine the eye structures for both eyes. The slit lamp exam will be performed prior to the paracentesis, and will evaluate lids, lashes, lens, conjunctiva, cornea, anterior chamber, pupils, cataract status and anterior vitreous.
4. Dilated indirect ophthalmoscopy will be performed to examine the retina of each eye after the pupils have been sufficiently dilated. The dilated indirect exam will evaluated the retinal vessels, macula, fovea, peripheral retina, optic nerve and mid/posterior vitreous.
5. Optical Coherence Tomography (OCT) will be performed prior to the paracentesis on both eyes and will be assessed to determine the central foveal thickness and macular volume.

A paracentesis will be performed at each study visit prior to anti-VEGF treatment. A topical anesthetic such at lidocaine, will be applied to the eye. A lid speculum will be inserted under the eyelids to hold the eye open during the paracentesis and the intravitreal injection. An application of betadine solution will be applied to the inferior sclera where a 30 gauge needle attached to a syringe will be injected into the eye to remove 0.1 mL of anterior chamber fluid. Following the paracentesis, while the speculum is still in place, the intravitreal injection of aflibercept will occur 4 mm behind the limbus inferiorly. Directly following the intravitreal injection, betadine will be applied to the injection site to minimize the risk of infection. All anterior chamber fluid samples will be stored until Spin Dx and ELISA analysis have been completed. The samples will be divided in the clinic.

All subjects will receive aflibercept starting on Day 1. Starting at week 4, macular thickness and VEGF levels will be evaluated to determine if treatment windows can be extended.

Pregnancy Testing and Reporting: If the subject is a female of childbearing potential, a urine pregnancy test will be performed prior to all study treatments unless it is determined that the subject is postmenopausal or is surgically sterile. Female subjects of childbearing potential will be required to use a reliable method of birth control while participating in this study. Reliable methods of birth control are: abstinence, oral contraceptives, OrthoEvra patch, NuvaRing, intrauterine devices (IUD), Nexplanon implant, DepoProvera, tubal ligation, or vasectomy of the partner (with confirmed negative sperm counts) in a monogamous relationship. An acceptable, although less reliable, method involves the careful use of condoms and spermicidal foam or gel and/or a diaphragm with spermicide with Plan B used for any noticed condom or diaphragm failures.

Study subjects will be told to inform the study doctor as soon as possible if pregnancy occurs during study participation. Any subject that becomes pregnant during study participation will be withdrawn from the study and asked to report the outcome of the pregnancy to the study doctor.

Contraceptive Measures for Males: Due to the unforeseen risks, sexually active male subjects will be required to use a medically acceptable form of birth control in order to be in this study. Medically acceptable contraceptives include: surgical sterilization such as a vasectomy or a condom used with a spermicide. Contraceptive measures such as Plan B are not acceptable methods for routine use. Male subjects will be asked to inform their female partners of the potential harm to an unborn child. If a female partner of a male study subject becomes pregnant the subject will be asked to notify the study doctor as soon as possible.

Risks of the Study: This study employs a paracentesis to obtain the fluid from the anterior chamber of the eye for the purpose of measuring the levels of VEGF, both by ELISA testing as well as the device under investigation, Spin Dx. Paracentesis is a process that is performed by a small (1%) minority of physician during a course of an anti-VEGF injection. The advantage of performing this process is to obtain this fluid (for this study) but also to lessen the risk of developing glaucoma which can occur in about 8% of patients without this step. The risks of performing a paracentesis is pain, bacterial endophthalmitis or intraocular infection, hyphema and inadvertent lens touch (4), hemorrhage, and a leak that might require temporary repair using Dermabond 2-Octyl Cyanoacrylate glue and a contact lens. The complications occur in less than 1 in 5000 injections.

Complications of the intravitreal injection procedure include endophthalmitis (5), or intraocular infection that occurs in less than 1 in 3000 injections, however can lead to blindness. Symptoms of endophthalmitis present with blurry vision, eye pain and red eye. The intravitreal injection procedure might cause a vitreous detachment, which can cause floaters, a retinal tear and bleeding into the vitreous. The rate of retinal detachment is 0 to 0.67% (5).

Aflibercept, administered through an intravitreal injection has the potential to cause arterial thromboembolic events (ATEs) (6). ATEs include stroke, myocardial infarction or vascular death. The incidence of these events is reported to be 1.8% in patients with wet age-related macular degeneration and 3.3% in patients with diabetic macular edema being treated with aflibercept (6). There have been no reports of thromboembolic events in the first 6 months of treatment of RVO (6). Intravitreal intraocular injections of aflibercept can cause an increase of intraocular pressure with acute increases typically occurring within 60 minutes of the injection (6). A sustained increase of intraocular pressure has been reported after repeated intravitreal injections of anti-VEGF (6). Additional adverse reactions in patients with RVO include eye pain, corneal epithelium defect, ocular hyperemia, foreign body sensation in treated eye and injection site pain (6).

Outcomes

### 1. Primary

a. Macular edema, measured by OCT, CST, change in mm
b. Macular volume, measured by OCT, change in cubic mm
c. VEGF levels, anterior chamber, measured by ELISA and Spin Dx

### 2. Secondary

a. Visual acuity, best corrected
b. IOP, in mm HG
c. Number of intravitreal injections

Analytical Plan

Pharmacodynamic studies will be done to create a model using the following variables:
1. VEGF levels, ELISA and Spin DX
2. OCT macular thickness, CST
3. OCT macular volume
4. VA, best corrected

### 5. Change in OCT CST

6. Change in OCT macular volume

### 7. Nonperfusion on IVFA

### REFERENCES

1. Walsh, David. A Centrifugal Fluidic Immunoassay for Ocular Diagnostics with an Enzyamatically Hydrolyzed Fluorogenic Substrate. Lab Chip, 2014, 14.2673-2680.
2. Campochiaro, P A, et al. Ranibizumab for Macular Edema Following BRVO: Six Month Primary End Point Results of a Phase III Study. Ophthalmology. 2011. 117, 1102-1112.
3. Brown, D M. Ranizumab for Macular Edema following CRVO. Six month Primary End Point of a Phase III Study. Ophthalmology. 2010; 117; 1124-30.
4. Helbig H, Noske W, Kleineidam M, Kellner U, Foerster M H; Bacterial endophthalmitis after anterior chamber paracentesis, Br J Ophthalmology 1995; 79:866
5. Falavarjani K G, Nguyen Q D. Adverse events and complications associated with intravitreal injection of anti-VEGF agents: a review of literature. Eye (Lond). 2013; 27(7):787-794. doi:10.1038/eye.2013.107
6. Eylea [package insert]. Tarrytown, N.Y.: Regeneron Pharmaceuticals, INC., 08/2019

What is claimed:

1. A method of treating an exudative maculopathy in subject comprising:
a) obtaining a sample of anterior chamber fluid from an anterior chamber of the subject's eye;
b) determining the level of vascular endothelial growth factor (VEGF) in the sample;
c) comparing the level of VEGF in the sample with a predetermined reference level of VEGF; and
d) administering to the eye of the subject intraocularly either an exudative maculopathy therapy A or an exudative maculopathy therapy B based on whether the level of VEGF in the sample is higher or not than the predetermined reference level therefor,
wherein if the level of the VEGF is higher than the predetermined reference level therefor then exudative maculopathy therapy A is administered and wherein if the level of the VEGF is not higher than the predetermined reference level therefor then exudative maculopathy therapy B is administered,
wherein exudative maculopathy therapy A is an anti-VEGF biologic or anti-VEGF small molecule, and
wherein exudative maculopathy therapy B is a either an anti-intercellular adhesion molecule 1 (ICAM1), anti-interleukin 8 (IL8), anti-interleukin 6 (IL6), anti-monocyte chemoattractant protein 1 (MCP1), anti-interleukin 1 alpha or beta (IL1 alpha/beta), anti-platelet-derived growth factor (PDGF), or anti-tumor necrosis factor alpha (TNF-alpha), or anti-interleukin-12 subunit p40 (IL12P40), and/or anti-angiopoietin (ANG2) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule, and
wherein steps b) through d) are performed within 2 hours of step a).

2. The method of claim 1, wherein the exudative maculopathy is a retinal vein occlusion and exudative maculopathy therapy B is a either an anti-intercellular adhesion molecule 1 (ICAM1), anti-interleukin 8 (IL8), anti-interleukin 6 (IL6), anti-monocyte chemoattractant protein 1 (MCP1) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

3. The method of claim 1, wherein the exudative maculopathy is a diabetic retinopathy and exudative maculopathy therapy B is a either an anti-intercellular adhesion molecule 1 (ICAM1), anti-interleukin 6 (IL6), anti-monocyte chemoattractant protein 1 (MCP1), anti-interleukin 1 alpha or beta (IL1 alpha/beta), anti-tumor necrosis factor alpha (TNF-alpha), and/or anti-angiopoietin (ANG2) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

4. The method of claim 3, wherein the subject is experiencing macular bleeding and the treatment reduces the bleeding.

5. The method of claim 1, wherein the exudative maculopathy is an exudative age-related macular degeneration and exudative maculopathy therapy B is a either an anti-platelet-derived growth factor (PDGF), anti-interleukin-12 subunit p40 (IL12P40), anti-intercellular adhesion molecule 1 (ICAM1), anti-monocyte chemoattractant protein 1 (MCP1) biologic or small molecule, or, wherein the subject has at least one week prior to step a) been administered an intraocular dose of an anti-VEGF biologic or anti-VEGF small molecule, therapy B is a lower dose of the anti-VEGF biologic or anti-VEGF small molecule.

6. The method of claim 5, wherein the subject is experiencing macular leakage and the treatment reduces the leakage.

7. A method of treating an exudative maculopathy in subject comprising:
i) obtaining a sample of anterior chamber fluid from an anterior chamber of the subject's eye;
ii) determining the level of one or more of the following molecules:
vascular endothelial growth factor (VEGF),
intercellular adhesion molecule 1 (ICAM1),
interleukin 6/8 (IL6/8),
monocyte chemoattractant protein (MCP1),
interleukin 1 alpha or beta (IL1 alpha/beta),
platelet-derived growth factor (PDGF),
tumor necrosis factor alpha (TNF-alpha),
interleukin-12 subunit p40 (IL12P40),
angiopoietin (ANG2),
iii) comparing the level of the one or more molecules with a predetermined reference level for each, respectively,
iv) administering to the eye of the subject intraocularly either an exudative maculopathy therapy A or an exudative maculopathy B based on whether the level of the one or more molecules is higher or not than the predetermined reference level for each, respectively, wherein if the level of the one or more molecules is higher than the predetermined reference level therefor then exudative maculopathy therapy A is administered and wherein if the level of the one or more molecules is not higher than the predetermined reference level therefor then exudative maculopathy therapy B is administered,
wherein when the molecule is VEGF then therapy A is an anti-VEGF biologic or anti-VEGF small molecule,
and wherein therapy B is a either an anti-intercellular adhesion molecule 1 (ICAM1), anti-interleukin 6/8 (IL6/8), anti-monocyte chemoattractant protein (MCP1), anti-interleukin 1 alpha or beta (IL1 alpha/beta), anti-platelet-derived growth factor (PDGF), anti-tumor necrosis factor alpha (TNF-alpha), anti-interleukin-12 subunit p40 (IL12P40), and/or anti-angiopoietin (ANG2) biologic or small molecule, or, wherein the subject has previously been administered a dose of an anti-VEGF biologic or anti-VEGF small molecule, is a lower dose of the anti-VEGF biologic or anti-VEGF small molecule, wherein steps ii) through iv) are performed within 2 hours of step i).

8. The method of claim 1, wherein the level of the VEGF or of the molecule in the sample is determined using a rapid ELISA.

9. The method of claim 1, wherein the level of the VEGF or of the molecule in the sample is determined using a rapid centrifugal fluidic immunoassay.

10. The method of claim 1, wherein steps b) through d) are performed within 1 hour of step a).

11. The method of any of claim 1, wherein steps b) through d) are performed within 0.5 hours of step a).

12. The method of claim 7, wherein steps ii) through iv) are performed within 1 hour of step i).

13. The method of claim 7, wherein steps ii) through iv) are performed within 0.5 hours of step i).

14. The method of claim 2, wherein the retinal vein occlusion is branch retinal vein occlusion (BRVO).

15. The method of claim 2, wherein the retinal vein occlusion is central retinal vein occlusion (CRVO).

16. The method of claim 1, wherein the intraocular administration is by intravitreal injection.
